(19) United States
(12) Patent Application Publication
(10) Pub. No.: US 2010/0280364 A1
(43) Pub. Date: Nov. 4, 2010

(54) NEAR REAL-TIME VIEWER FOR PET-GUIDED TISSUE INTERVENTIONS

(75) Inventors: Xiaohong Lu, Del Mar, CA (US); Stephen T. Yarnall, Poway, CA (US)

Correspondence Address: KATTEN MUCHIN ROSENMAN LLP;(C/O PATENT ADMINISTRATOR), 2900 K STREET NW, SUITE 200, WASHINGTON, DC, 20007-5118, US

(73) Assignee: Naviscan, Inc., San Diego, CA (US)

(21) Appl. No.: 12/769,236

(22) Filed: Apr. 28, 2010

Related U.S. Application Data
(63) Continuation of application No. 12/603,203, filed on Oct. 21, 2009, now abandoned.
(60) Provisional application No. 61/107,607, filed on Oct. 22, 2008.

(51) Int. Cl. A61B 6/12

(52) U.S. Cl. 600/424

(57) ABSTRACT

An apparatus and a computational method are provided for localizing a positron-emitting source during an intervention using positron emission tomography (PET) imaging. The apparatus is designed to accelerate the image acquisition and reconstruction process such that the resulting image presentation can be used to quickly reposition an interventional device in relation to a lesion. By limiting the processing of data in the acquisition to a focal plane which extends through the localization target, and which is oriented to capture and display motion during the intervention, and then by displaying a persistence image which is frequently refreshed, a user can visualize movement of the positron-emitting source for optimizing the position of the interventional device. The positron-emitting source(s) can be radiolabeled tissue, a radiolabeled interventional device, a radiolabeled fiducial, or any combination thereof.

### CROSS REFERENCE TO RELATED APPLICATIONS

The application is a continuation of U.S. patent application Ser. No. 12/603,203, filed Oct. 21, 2009, and claims priority benefit to Provisional U.S. Patent Application No. 61/107,607, filed Oct. 22, 2008, the contents of which are incorporated herein in their entirety.

### BACKGROUND OF THE INVENTION

1. Field of the Invention

The present invention relates generally to an apparatus and method for positioning interventional devices using PET imaging. More particularly, the present invention relates to a system and method for reducing the time needed to track and display the physical relationship between an interventional device and a lesion as the interventional device is being applied.

2. Description of Related Art

Cancer is a major threat and concern to the population. Early detection and complete treatment of suspicious or cancerous lesions has been shown to improve long-term survival. Ex-vivo imaging modalities such as magnetic resonance imaging (MRI), x-ray, and ultrasound are often conventionally deployed to detect small, non-palpable lesions. Once detected, a tissue sample (biopsy) from the lesion is obtained using position information from one or more of these conventional ex-vivo imaging modalities. The tissue sample is then analyzed for the presence of cancer to determine if the lesion requires treatment. If the lesion is found to require treatment (e.g., excision, ablation, radiation), position information from an ex-vivo imaging modality is sometimes used to localize the borders of the lesion so not more nor less tissue than necessary is treated.

There are a myriad of devices, in practice or proposed, to mark, sample (biopsy), and treat (e.g., excision, ablation, radiate, or poison) suspicious or cancerous tissue using ex-vivo image localization. Each of these devices produces a signal that can be detected by one or more ex-vivo imaging modalities. This signal can be used to ensure the device has been positioned properly in relation to the suspect tissue.

One example of a marking device and method is the common wire-localized biopsy, where an x-ray-opaque guide wire is used to localize non-palpable lesions detected by x-ray or ultrasound for subsequent biopsy or excision. A hollow needle with an open, sharpened tip is inserted percutaneously, into or near the suspect tissue, based on x-ray or ultrasound positioning. The guide wire, with a spring-loaded anchoring hook at its tip, is then introduced through the needle and advanced until the anchoring tip projects out the distal end of the needle, at which point the hook is deployed resisting backward displacement of the wire. The needle is then withdrawn, leaving the guide-wire in the desired position. The final position of the wire with respect to the lesion is determined and confirmed with subsequent x-ray or ultrasound views. The result of this procedure is then used in surgery as a physical representation of the position of the lesion to guide a biopsy or excision. It is clearly critical that the positioning of the guide wire(s) accurately depict the position of the lesion to ensure that a proper tissue sample is obtained for analysis and/or to ensure that the borders of the lesion are accurately represented for a complete excision of the lesion with minimal complications, scarring and deformity.

An example of a sampling or biopsy device and method used to localize non-palpable lesions detected by ex-vivo imaging is the common image-guided core-biopsy needle procedure. The core biopsy needle is a minimally-invasive tissue sampling device that can be introduced percutaneously into suspect tissue based on x-ray, ultrasound, or MRI positioning. The needle has an aperture or sampling window for capturing and removing tissue after its position in relation to the lesion has been established by x-ray, ultrasound or MRI imaging. It is clearly critical that the position of the sampling window is within, or directly adjacent to the suspect tissue to ensure a proper sample is obtained for analysis.

Albrecht, et al. discloses an example of a method and device to treat cancerous lesions by excision in U.S. Pat. No. 6,840,948 and U.S. Pat. No. 6,855,140, the contents of both of which are incorporated herein by reference in their entireties. These patents describer a means for the intact removal of a lesion under image guidance. A rotatable electrode is inserted into the tissue and positioned adjacent to the lesion, such that by rotationally driving the electrode, it envelops the lesion, severing it from the surrounding tissue for intact removal. Ex-vivo imaging is used to assist in placement of the probe, and to assess a desired excision volume. To ensure a complete removal of the cancerous tissue using this method, it is clearly critical to position the electrode directly adjacent to the lesion and confirm the placement with ex-vivo imaging before the excision.

The effectiveness of each of these methods relies on the accuracy of ex-vivo image localization, including x-ray, ultrasound, MRI, and positron emission tomography (PET) to delineate suspect tissue and to describe the position of the device in relation to that delineation. Thus, the success of the localization and the ensuing procedures relying on that localization are strongly related to the accuracy of the imaging modality.

Further affecting the success of the localization is the ability to track the progress of the intervention in clinically-suitable timeframes such that correction to the placement or trajectory of the interventional device can be made promptly, thus avoiding unacceptable delays in completing the procedure. For example, handheld ultrasound probes are routinely used to visualize the placement of an interventional device in relation to a sonographic target. X-ray fluoroscopy is used to guide interventions of x-ray visible targets. These modalities share the common characteristic of being suitable for tracking the progress of an intervention in near-real time. However, neither of these modalities allows the ability to track the progress of the intervention with respect to the changes in cellular functional which may be associated with neoplasia and is therefore important for interventional guidance.

Nuclear medicine techniques have been adapted for measuring biochemical functions in the human body. One of these methods, positron emission tomography (PET), involves the detection of gamma rays emitted from tissues after administration of a substance such as glucose or fatty acids into which positron emitting isotopes (e.g., radiotracers) have been incorporated. A computer algorithm interprets the paths of the gamma rays that result from collisions of positrons and electrons, and the resultant tomogram represents the distribution of the isotope within the imaged tissue.

Among the ex-vivo imaging modalities, PET is unique because it produces images of the body's basic biochemistry or function. Traditional diagnostic techniques, such as x-rays, computerized axial tomography (CT) scans, and MRI, produce images of the body's anatomy or structure. The premise with these techniques is that the change in structure or anatomy that occurs with disease can be seen.

Biochemical processes are also altered with disease and may occur before there is a detectable change in gross anatomy. PET is an imaging technique that is used to visualize some of these processes that change. Even in diseases such as Alzheimer's disease, where there is no gross structural abnormality, PET is able to show a biochemical change. PET is a very useful addition to the clinician's diagnostic toolbox, providing significant advances to traditional diagnostic methods.

In cancer imaging, PET using the administration of the radiotracer flourodeoxyglucose (FDG-PET) is a method of measuring the rate of glucose metabolism within tissue. Increased glucose metabolism is often associated with neoplastic processes. FDG-PET is becoming standard in clinical diagnostic practice, as increased glucose metabolism is one of the earliest ex-vivo methods of cancer detection.

A limitation for performing interventions using nuclear-emission localization such as FDG-PET is the inability to simultaneously image a region of suspicious tissue together with the interventional device for completely describing their relative positions, because the devices do not produce a signal that is detectable by nuclear-emission imaging techniques. Thus, a method for making the device produce a signal that is detectable by nuclear-emission imaging (e.g., FDG-PET) for the purpose of describing the orientation and location of the device has been disclosed in U.S. Patent Application Publication No. U.S. 2007/0167749, the contents of which are incorporated herein by reference in their entirety.

As described above, the accuracy of orienting a device in proper relation to the suspect tissue is critical to the success of the ensuing procedure. A recently-developed PET scanner (i.e., PEM Flex, Naviscan Inc.) designed for organ-specific imaging, provides adequate resolution (e.g., 2 mm in-plane) for detecting small abnormalities as well the ability to compress and immobilize the volume of tissue being imaged with respect to the detector system. These features make PET-guided interventions possible, provided that the time to acquire, reconstruct, and display the images is clinically reasonable.

However, a current shortcoming of PET scanners is the time required to acquire and display an image for tracking the application of the interventional device. Typical PET studies require a clinically-significant time (e.g., 5-15 minutes) to acquire, reconstruct, and display an image, greatly extending the duration of a PET-guided intervention when compared with other modalities used to guide interventions such as ultrasound or x-ray fluoroscopy.

Accordingly, a method of PET imaging that allows the clinician to quickly (i.e., as with fluoroscopy or ultrasound) visualize the position of an interventional device in relation to a lesion is needed to in order to allow PET-guided interventions in clinically-appropriate time frames.

Additional relevant prior art references include the following, each of which is incorporated herein by reference in its entirety: U.S. Pat. No. 6,740,882 to Weinberg discloses a method using PET to guide interventions comprising detecting gamma radiation emitted from a patient and using the detected gamma radiation to determine stereotactic data. The detecting mechanism includes a pair of detector modules disposed one on each side of an immobilizing mechanism.

Raylman, et al., “Positron Emission Mammography—Guided Breast Biopsy”, The Journal of Nuclear Medicine, Vol. 42, No. 6, pages 960-966, June 2001, discloses a method of marking the tip of a biopsy needle with a small, sealed positron-emitting point source for the purposes of verifying the position of an interventional device in a PET-guided stereotactic procedure.

U.S. Patent Application Publication No. U.S. 2007/0167749 to Yarnall, et al. discloses a method of marking of an interventional device with a position emitting source such that its position and orientation relative to a lesion can be determined for accurately guiding an intervention using PET imaging.

Beylin, et al., RSNA 2005 Presentation, “Fluoroscopic High Resolution PET Guidance for Breast Biopsy and Interventions; High Resolution PET Guidance for Breast Biopsy”, describes continuous visualization (5 second frames) of an intervention in a PET-guided intervention as “currently not feasible with physiologic levels of FDG uptake: hard to visualize lesions on 5 sec PEM images.”

The primary shortcoming of the combined, described prior art is the ability to utilize a near-real time tracking technique while applying an interventional device, which may or may not be labeled with a radioactive marker, in order to optimally localize a lesion during a PET-guided intervention.

### SUMMARY OF THE INVENTION

The current invention comprises a method and apparatus for rapid acquisition, processing, and display of PET images used for visualizing and tracking PET-guided interventions.

Accordingly, in one aspect, the present invention provides a method for localizing a positron-emitting source during an intervention relating to a body part. The method comprises the steps of: injecting a predetermined amount of a radiotracer into the body part; immobilizing and compressing the body part; acquiring a diagnostic image of the body part using positron emission tomography; using the acquired image to determine a location of a lesion within the body part; positioning at least two detectors such that the determined lesion location is within a field of view of each of the at least two detectors; obtaining a persistence image of the lesion within a predetermined focal plane using the positioned detectors; applying a radiolabeled interventional device in the focal plane; and performing an intervention using the applied device and the obtained persistence image. The step of obtaining a persistence image may include using the positioned detectors to obtain image data relating to the lesion within the predetermined focal plane over a predetermined time interval. The predetermined time interval may be user-adjustable. The method may further include the steps of displaying the obtained persistence image and periodically refreshing a display of the persistence image. The step of periodically refreshing the display of the persistence image may further include defining a refresh rate, the refresh rate being user-adjustable. The method may further include the step of using a refreshed display of the persistence image to adjust a trajectory of the applied device.

In another aspect, the invention provides a system for localizing a positron-emitting source during an intervention relating to a body part. The system comprises a positron emission tomography (PET) scanner having at least two detectors and a display monitor. The PET scanner is configured to enable a user to acquire a diagnostic image of the body part and to use the acquired image to determine a location of a lesion within the body part; and to further enable the user to position the at least two detectors such that the determined lesion location is within a field of view of each of the at least two detectors, and to obtain a persistence image of the lesion within a predetermined focal plane using the positioned detectors. The display monitor is configured to display the obtained persistence image, and to enable the user to apply a radiolabeled interventional device in the focal plane, and to perform an intervention using the applied device and the obtained persistence image. The PET scanner may be further configured to obtain a persistence image by obtaining image data relating to the lesion within the predetermined focal plane over a predetermined time interval. The predetermined time interval may be user-adjustable. The display monitor may be further configured to periodically refresh the display of the persistence image based on a user-adjustable refresh rate and to further enable the user to adjust a trajectory of the applied device using the refreshed display.

### BRIEF DESCRIPTION OF THE DRAWINGS

FIG. 1 illustrates a patient configured in a breast-optimized PET scanner.

FIG. 2 illustrates a breast being compressed and immobilized in a breast-optimized PET scanner.

FIG. 3 illustrates a limited field image acquisition in comparison with a regional image acquisition.

FIG. 4 illustrates a diagram of focal plane for a breast-optimized PET scanner.

FIG. 5 illustrates a derivation of a focal plane.

FIG. 6 illustrates a focal plane image of a radioactive line source.

FIG. 7 illustrates a sequence of persistence images without background.

FIG. 8 illustrates a sequence of persistence images with background.

FIG. 9 illustrates a flowchart showing procedural elements for a method of localizing a positron-emitting source during an intervention using PET imaging, according to a preferred embodiment of the invention.

### DETAILED DESCRIPTION OF THE INVENTION

A preferred embodiment of the invention includes the subject method used in conjunction with a high-resolution PET scanner capable of compressing and immobilizing target tissue labeled with a positron-emitting radiopharmaceutical (e.g., FDG) for a stereotactic or manually-guided intervention. A second preferred embodiment includes the additional element of a radioactive marker affixed to the interventional device such that its position can be detected with the PET scanner. A third preferred embodiment involves the use of the method to determine the position and orientation of the interventional device relative to the lesion.

In a typical PET scan, there are very large number of different coincidence lines (also commonly referred to as lines of response, or LORs) through the three dimensional space between the opposing detector modules. A sufficient number and quality of LORs (and the associated time to acquire them) are necessary to provide good statistical data for image clarity. Once the LORs have been acquired, processing the LORs into a suitable image typically involves the application of sophisticated rebinning (e.g., Fourier) and reconstruction algorithms (e.g., maximum likelihood expectation maximization, or MLEM) that require substantial computational time and make near-real time tracking during an intervention unfeasible.

It is therefore an object of the present invention to provide a method and apparatus for limiting the acquisition, rebinning, and reconstruction time needed to produce and present images in a way that is suitable for tracking a PET-guided intervention in near real-time. In these respects, the described invention departs from the existing concepts and designs. These and other objectives are met with an embodiment of the present invention as described below.

Other objects, features and advantages of the present invention will become apparent to those skilled in the art from the following detailed description. It is to be understood, however, that the detailed description and specific examples, while indicating preferred embodiments of the present invention, are given by way of illustration and not limitation. Many changes and modifications within the scope of the present invention may be made without departing from the spirit thereof, and the invention includes all such modifications.

### A Near Real-Time Viewer For PET-Guided Interventions

A preferred embodiment of a near real-time viewer for PET-guided interventions comprises an adaptation of the normal image acquisition, reconstruction and display method used in positron emission tomography PET imaging for producing an image which is suitable for guiding interventions in near real time. This method may include of one or more of the following steps:

1) Limiting the acquisition of data to only the region necessary to visualize the intended intervention.

2) Limiting the processing of data in the acquisition to a focal plane which extends through the localization target, and which is oriented to capture and display motion during the intervention. This will generally be parallel to the direction of the intervention, however it could be any plane that can be utilized to track the movement of any of the radiolabeled elements.

3) Displaying a “persistence” period of temporally processed image data from memory.

4) Refreshing the display in near real-time for tracking the progress of the intervention.

5) Utilizing high resolution PET imaging with compression and immobilization.

6) A method that can be used to determine the physical relationship between a lesion and a radiolabeled device in both position and orientation.

7) A method that can be applied to a variety of device configurations that are design to be utilized in-vivo (e.g., cannula, percutaneous tissue extraction device, brachytherapy seed introducer, biopsy site marker).

### Immobilizing the Body Part

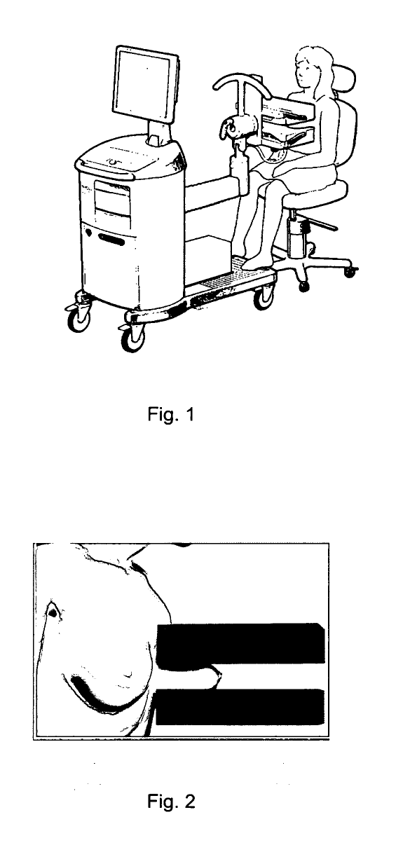

A drawing of a patient positioned in a PET scanner is shown in FIG. 1. In this case, the patient's breast is being imaged in a PET scanner that has been designed for breast imaging and intervention (e.g., PEM Flex, Naviscan Inc.). In a normal diagnostic scan, the pair of moveable detectors is scanned in unison across the field of view to acquire a full-breast image.

FIG. 2 shows a side view of a breast that has been positioned for imaging in a in a PEM Flex scanner. Patient trays or “paddles” hold the breast in compression in order to immobilize it in detector space. Likewise, in a whole-body PET scanner, immobilization of the region is necessary to maintain detector to tissue co-registration to optimally apply the near real-time viewer.

### Reduce Acquisition Time

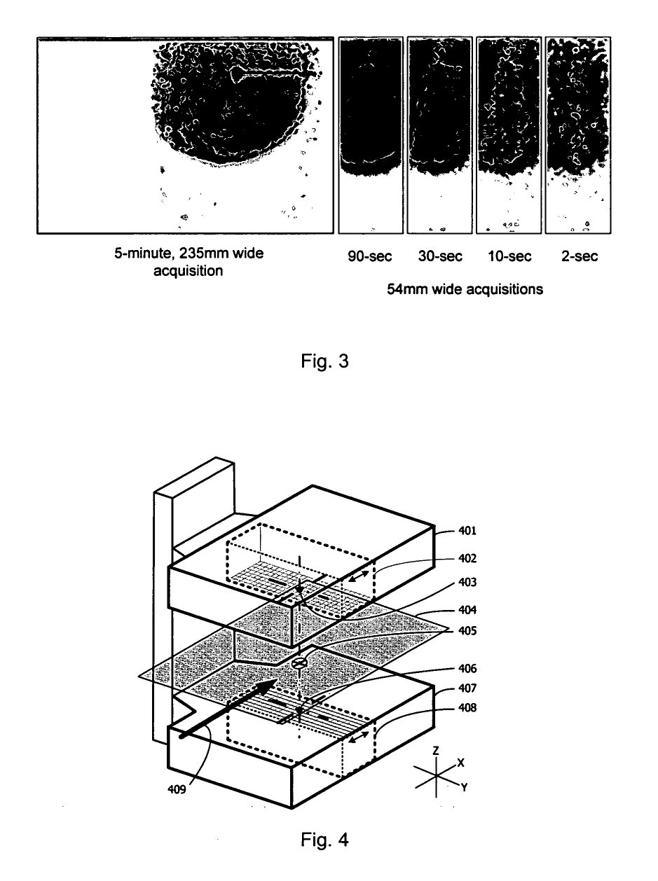

For use with the near real-time visualization tool, the moveable detectors are positioned or “parked” directly over the interventional area (i.e., over the lesion) during the acquisition, thus constraining the field of view and reducing the acquisition time when compared to a standard diagnostic scan of the region, which is typically 10 minutes or more. Likewise, in a whole body PET scanner, instead of scanning the entire thorax for a lung intervention, the gantry would be fixed in position, with the region for the intervention positioned axially within the detector rings. FIG. 3 shows limited-field acquisitions containing 90, 30, 10, and 2 seconds of image data compared to a 5 minute acquisition of the full field. Note that a useable image for interventional guidance is produced in 10 seconds in this clinically-realistic case consisting of a 6:1 lesion to background ratio with a radiolabeled line source tip near a breast lesion.

### Reduce Image Processing Time

In order to eliminate the need for a time-consuming three-dimensional reconstruction (e.g., MLEM, OSEM), acquisition data is recorded onto a “focal plane” which passes through the region intended for intervention. The focal plane is determined by the user from cross sectional diagnostic scan images. It should be parallel to the direction desired for the intervention and it should pass through the interventional target (e.g., lesion). For example, for a lesion located at coordinates x, y, z in detector space, and for an intervention that is taking place in the in the x-direction, the focal plane could be the x-y plane passing through point z, or the x-z plane passing through point y. To optimize real-time visualization, the focal plane also coincides with the highest spatial-resolution imaging plane provided by the PET scanner.

FIG. 4 shows an example of a focal plane for a breast intervention as represented in the breast-optimized system described above. The breast is immobilized between compression paddles 401 and 407. Moveable detectors 402 and 408 are positioned over the center of the interventional target 405 as shown by points labeled 403 and 406 on the face of the detectors. The x-y focal plane 404 slices through the interventional target 405 with the direction of motion for the intervention shown as 409.

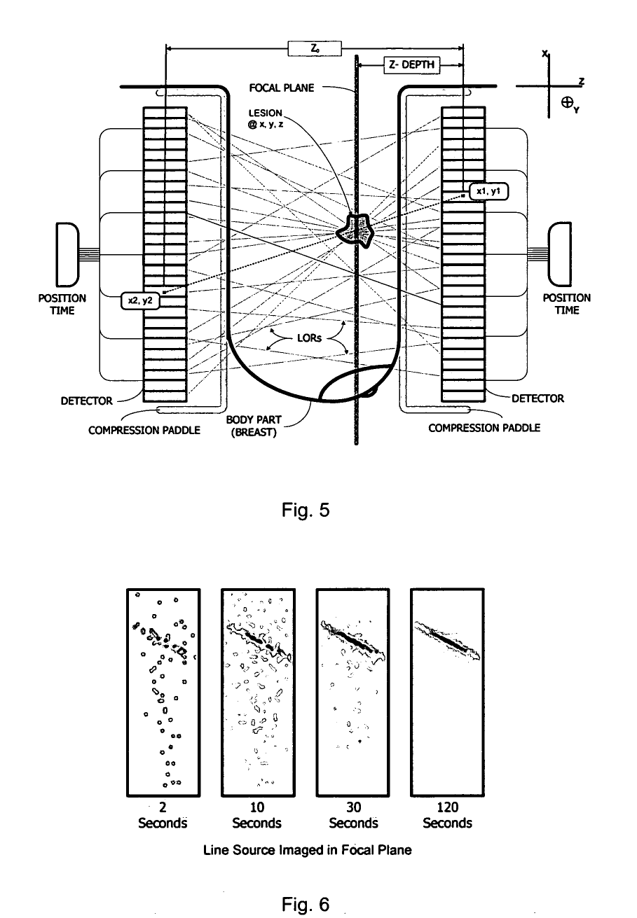

As shown in FIG. 5, the intersection point of each line-of-response (LOR) through the focal plane is calculated using the x-y coordinates (x, y) given by the vector equation:

(x, y)=(x₁, y₁)+Z_{depth}/Z₀*(x₂=x₁, y₂=y₁),

where Z₀ is the separation between the average location for the gamma interaction within the crystals from opposing detectors.

FIG. 6 shows an image of a radioactive line source using data from the defined focal plane. Note that the image clarifies as the acquisition time, and resulting number of LORs, increases. In order to track the application of a radiolabeled interventional device, the radioactive line source must be applied in the focal plane or else the image may appear blurred. Accordingly, blurring is an indication that the position of the radioactive source is not within the focal plane, which is information that can be used for targeting in all three dimensions. Although the line source is much longer than the image shown in FIG. 6, the sensitivity of the detector is maximized at its center with the corresponding improvement in image clarity near the center.

### Image Display

Images are comprised of data collected for a “persistence period.”Each time the display is refreshed, a new image is displayed, corresponding with the data acquired during the most recent persistence period. The persistence period is preferably user-adjustable, to adapt for variations in motion and counting statistics. For example, longer persistence periods will generally produce images with increased motion blurring, and images from shorter periods will appear more grainy due to a lack of counts. In addition, various filters can be applied to the persistence images to optimize image clarity (e.g., adaptive temporal filters; see, for example, U.S. Pat. No. 4,887,306 to Hwang, et al.) and to reduce the effects of motion blurring. The optimal persistence time is determined based upon a balance between image quality (sufficient counts), the speed of moving objects, and the desired reduction of tailing artifacts.

In the real time viewer, the display is frequently refreshed with the latest persistence image in order to allow visualization of motion. A longer time interval between display refreshes can help to accentuate to appearance of motion; however, a shorter interval will more accurately denote the real-time position of the radioactive elements in to the focal plane. The refresh interval is preferably user-adjustable and it can be less than, equal to, or greater than the persistence period.

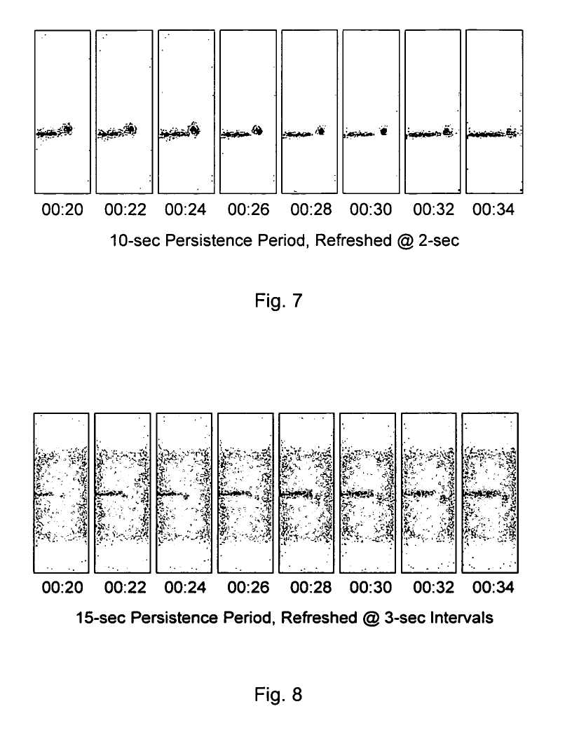

FIG. 7 shows 10-second persistence images of a line source pushing a simulated lesion to the right. Note the clarity of the images in the absence of realistic background. As shown in FIG. 8, clinically realistic lesion to background ratios (e.g., 4:1), will require an increase in the persistence period in order to obtain adequate image clarity.

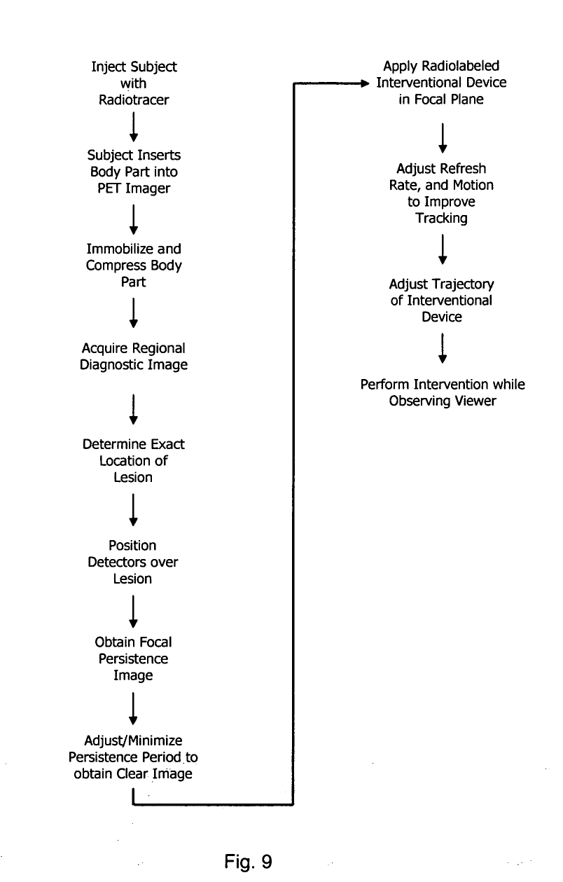

FIG. 9 is a flowchart that illustrates procedural elements involved in an intervention using the near real-time viewer according to a preferred embodiment of the present invention.

Alternatively, embodiments of the present invention may be utilized at a variety of anatomical sites, including tissue removal sites, biopsy sites (e.g., lung, prostate, liver), polyp sites, lesion sites, or other sites of interest.

While the foregoing detailed description has described particular preferred embodiments of this invention, it is to be understood that the above description is illustrative only and not limiting of the disclosed invention. For example, the method of the present invention may also be carried out by observing the progress of harvesting radiolabeled tissue as might be desirable during a biopsy or excision. While preferred embodiments of the present invention have been shown and described herein, it will be obvious to those skilled in the art that such embodiments are provided by way of example only. For example, use of the technique during freehand interventions where the tissue is not completely immobilized in detector space is practical, provided sufficient count rates are available to fill the persistence data buffers in a clinically-appropriate timeframe. Numerous variations, changes, and substitutions will now occur to those skilled in the art without departing from the invention. For example, robotically-controlled surgery could be guided by near real-time images where the radioactive targets are digitally ‘tracked’ during the intervention and fed back to the motion controller.

What is claimed is:

1. A method for localizing a positron-emitting source during an intervention relating to a body part, the method comprising the steps of:
immobilizing and compressing the body part;
acquiring a diagnostic image of the body part using positron emission tomography;
using the acquired image to determine a location of a lesion within the body part;
positioning at least two detectors such that the determined lesion location is within a field of view of each of the at least two detectors;
obtaining a persistence image of the lesion within a predetermined focal plane using the positioned detectors;
applying a radiolabeled interventional device in the focal plane; and
performing an intervention using the applied device and the obtained persistence image.

2. The method of claim 1, wherein the step of obtaining a persistence image further comprises using the positioned detectors to obtain image data relating to the lesion within the predetermined focal plane over a predetermined time interval.

3. The method of claim 2, wherein the predetermined time interval is user-adjustable.

4. The method of claim 1, further comprising the steps of displaying the obtained persistence image and periodically refreshing a display of the persistence image.

5. The method of claim 4, wherein the step of periodically refreshing the display of the persistence image further comprises defining a refresh rate, the refresh rate being user-adjustable.

6. The method of claim 4, further comprising the step of using a refreshed display of the persistence image to adjust a trajectory of the applied device.

7. A system for localizing a positron-emitting source during an intervention relating to a body part, the system comprising a positron emission tomography (PET) scanner having at least two detectors, and a display monitor,
wherein the PET scanner is configured to enable a user to acquire a diagnostic image of the body part and to use the acquired image to determine a location of a lesion within the body part; and to further enable the user to position the at least two detectors such that the determined lesion location is within a field of view of each of the at least two detectors, and to obtain a persistence image of the lesion within a predetermined focal plane using the positioned detectors; and
wherein the display monitor is configured to display the obtained persistence image, and to enable the user to apply a radiolabeled interventional device in the focal plane, and to perform an intervention using the applied device and the obtained persistence image.

8. The system of claim 7, wherein the PET scanner is further configured to obtain a persistence image by obtaining image data relating to the lesion within the predetermined focal plane over a predetermined time interval.

9. The system of claim 8, wherein the predetermined time interval is user-adjustable.

10. The system of claim 7, wherein the display monitor is further configured to periodically refresh the display of the persistence image based on a user-adjustable refresh rate and to further enable the user to adjust a trajectory of the applied device using the refreshed display.
